(19) United States
(12) Plant Patent Application Publication
(10) Pub. No.: US 2014/0090120 P1
(43) Pub. Date: Mar. 27, 2014

(54) Cherry rootstock plant named 'STO3'

(50) ;

(71) Applicant: Peter Stoppel Obstbau, Kressbronn (DE)

(72) Inventor: Peter Stoppel, Kressbronn (DE)

(21) Appl. No.: 13/987,743

(22) Filed: Aug. 27, 2013

(30) Foreign Application Priority Data
Sep. 24, 2012 (EP) 2012/2007

(51) Int. Cl. A01H 5/00

(52) U.S. Cl. PLT/183

(57) ABSTRACT

A new and distinct cultivar of Prunus plant named ‘STO3,’ characterized by its upright plant habit and typical hybrid growth; good adaptation to hot and dry conditions; and as a rootstock, weak vegetative growth imparts uniformity, higher yield and better fertility to the grafted variety.

### RELATED APPLICATION INFORMATION

This application claims priority to European Community Plant Variety Application No. 2012/2007, filed Sep. 24, 2012; the disclosure of which is incorporated herein by reference in its entirety.

### LATIN NAME OF THE GENUS AND SPECIES

The Latin name of the genus and species of the plant variety disclosed herein is Prunus cerasus L.×P.×schmittii Rehder.

### VARIETY DENOMINATION

The inventive cultivar of Prunus cerasus L.×P.×schmittii Rehder disclosed herein has been given the varietal denomination ‘STO3.’

### BACKGROUND OF THE INVENTION

The present invention relates to a new and distinct cultivar of Prunus plant, typically used as a cherry tree rootstock, botanically known as P Prunus cerasus L.×P.×schmittii Rehder, and hereinafter referred to by the name ‘STO3.’

The new and distinct variety of cherry hybrid tree of the present invention was bred as a cross of Prunus cerasus with a Prunus canescens hybrid in Germany in 1989. It has been successfully asexually propagated by tissue culture. The purpose of breeding program was to find a better adapted cherry rootstock. The new Prunus plant is better adapted to heat and aridity than other rootstocks with a weak vegetative growth. The breeder has succeeded in creating a new cherry rootstock, which has a higher yield and influences the grafted variety positively in fertility.

### SUMMARY OF THE INVENTION

Plants of the new Prunus have not been observed under all possible environmental conditions. The phenotype may vary slightly with variations in environment such as temperature and light intensity without any variance in genotype.

The following traits have been repeatedly observed and are determined to be the unique characteristics of ‘STO3.’ These characteristics in combination distinguish ‘STO3,’ as a new and distinct cultivar of Prunus:

1. Upright plant habit;

2. Typical hybrid growth;

3. Good adaptation to hot and dry clime conditions;

4. As a rootstock, with moderate vegetative growth, it produces a dwarf tree;

5. As a rootstock, imparts uniformity and higher yield to the grafted variety;

6. 40% less vigour compared to Prunus avium seedlings; and

7. Absence of suckers.

Plants of the new Prunus can be compared to plants of Prunus ‘Weiroot 720’ (U.S. Plant Pat. No. 22,867, issued Jul. 24, 2012). In side-by-side comparisons conducted in Kressbronn, Germany, plants of the new Prunus differed primarily from plants of ‘Weiroot 720’ in the following characteristics:

1. As a rootstock, plants of the new Prunus have about double the growth in comparison to plants with ‘Weiroot 720’ rootstocks;

2. Plants of the new Prunus have hairy one-year-old shoots, whereas ‘Weiroot 720’ has a hairless one-year-old shoot;

3. Plants of the new Prunus have hairs on the lower surface from the leaves, whereas ‘Weiroot 720’ does not have any hairs on the lower surface from the leaves; and

4. Due to its typical hybrid growth, plants of the new Prunus impart higher yield to the scion plant than plants of ‘Weiroot 720.’

Asexual reproduction of Prunus rootstock plant ‘STO3’ by tissue culture since 2001 has shown that the unique features of this new variety are stable and the plant reproduces true to type in succeeding generations of asexual reproduction.

### BRIEF DESCRIPTION OF THE DRAWINGS

The accompanying photographs illustrate the overall appearance of the new Prunus plant. These photographs show the colors as true as it is reasonably possible to obtain in colored reproductions of this type. Colors in the photographs may differ slightly from the color values cited in the detailed botanical description which accurately describe the colors of the new Prunus plant.

FIG. 1 provides a side perspective view of a typical tree of ‘STO3’ grown in an outdoor orchard.

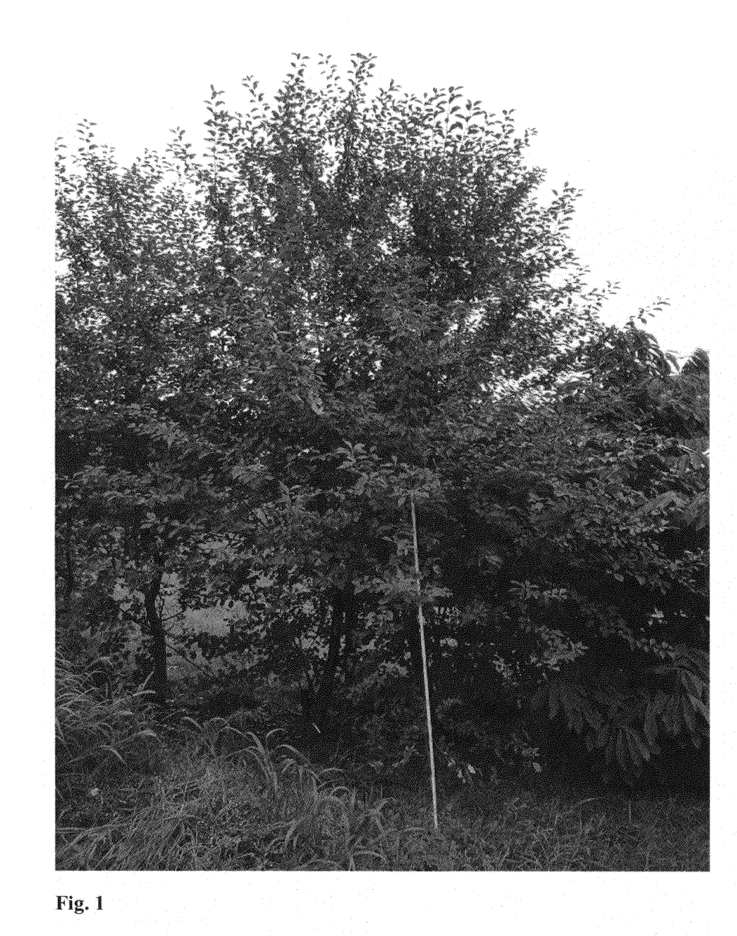

FIG. 2 provides a close-up view of leaves of ‘STO3’.

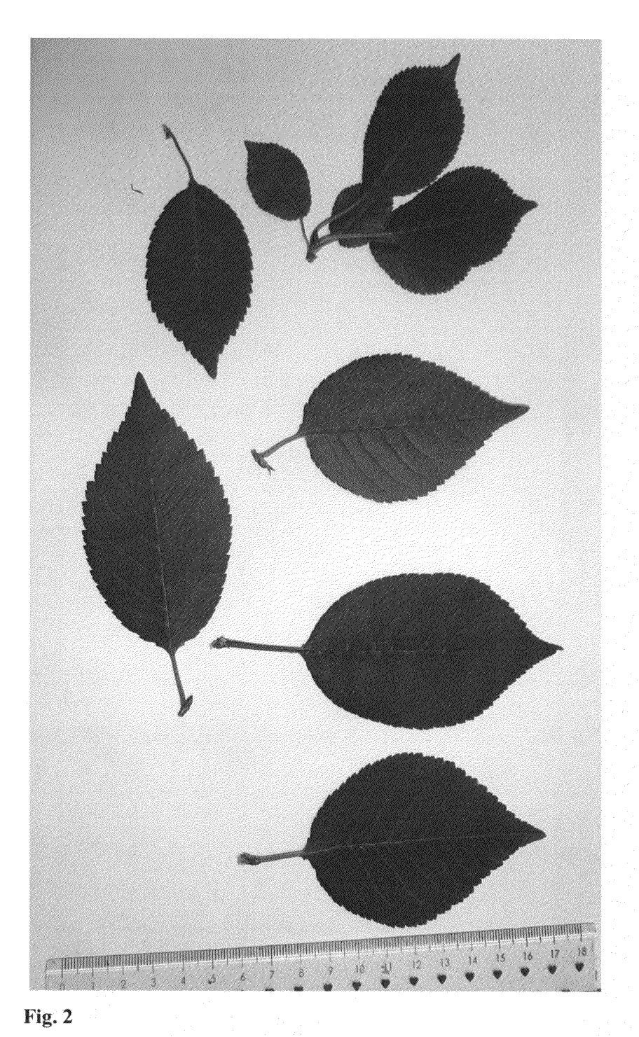

FIG. 3 provides a close-up view of fruits, branches and leaves of ‘STO3.’

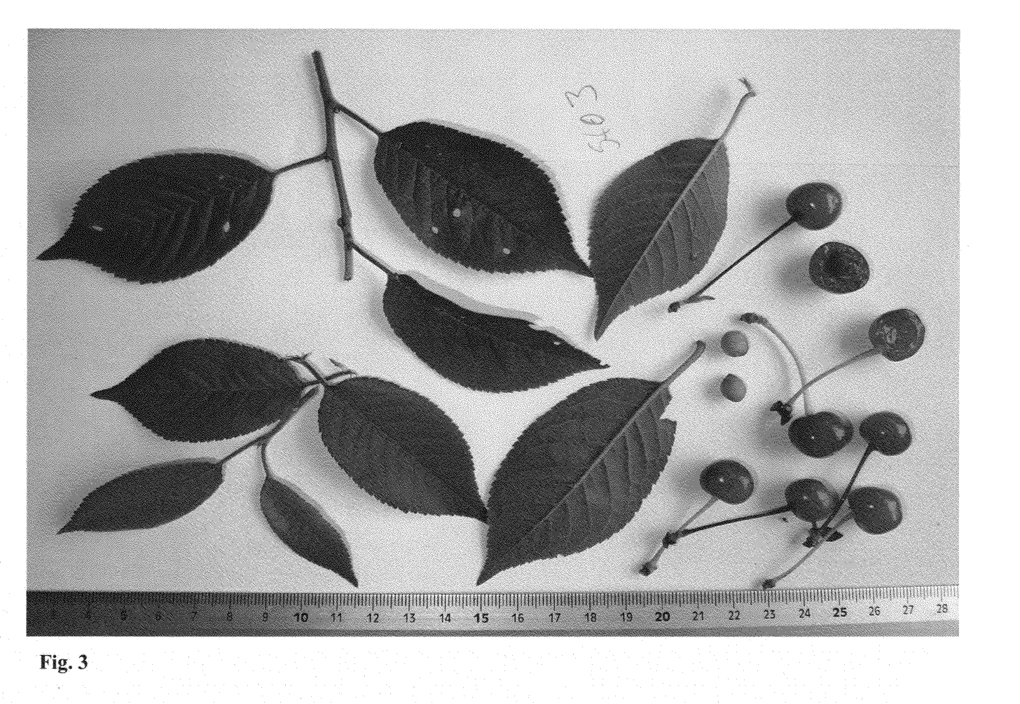

### DETAILED BOTANICAL DESCRIPTION

The aforementioned photographs, following observations and measurements describe plants grown during the summer in Kessbronn, Germany in an outdoor orchard and under conditions and practices generally used in commercial cherry production. Plants were 11 years old when the photographs and descriptions were taken. Measurements and numerical values represent averages for typical plants and plant parts. The actual measurements of any individual plant or plant parts, or any group of plants or plant parts, of the new Prunus plant may vary from the stated average. In the following description, color references are made to The Royal Horticultural Society (R.H.S.) Colour Chart, except where general terms of ordinary dictionary significance are used.

‘STO3’ is generally erect and medium strong in growth. The hybrid growth is typical for the new Prunus plant. After eleven growing seasons in Kressbronn, Germany, a height of about 5.6 meters, a width of about 3.5 meters and a trunk diameter of about 13 cm were observed for ‘STO3.’ The growth reduction induced by ‘STO3’ allows a highly intensive cherry production with dwarf trees.
Botanical classification: Prunus cerasus L.×P.×schmittii Rehder ‘STO3.’
Parentage: Breeding between Prunus cerasus and a Prunus canescens hybrid.
Propagation:
   Type.—By asexual reproduction.
   Method.—By tissue culture since the autumn of 2001 in a controlled environment. The process is similar to the propagation of other cherry rootstock.
Plant description:
   Plant and growth habit.—Used a rootstock; upright plant habit; plants are typically grown as a single stem; moderate vigor.
   Stem description.—Strength: Strong. Texture: Rough. Color: Grey brownish RHS 199B.
   Leaf description, fully developed leaves.—Arrangement: Alternate; simple. Length: About 5.8 cm to about 7.2 cm. Width: About 3.5 cm to about 4 cm. Shape: Elliptic to ovate, moderately elongated. Apex: Acute. Tip: Acuminate. Base: Obtuse. Margin: Biserrate. Texture, upper surface: Glabrous. Texture, lower surface: Coarse, pubescent. Color: Fully developed leaves, upper surface: Close to RHS 136A; venation, close to RHS 146C. Fully developed leaves, lower surface: Close to RHS 137C; venation, close to RSH 152D.
   Petiole description.—Length: About 1 cm to about 1.6 cm. Diameter: About 0.16 cm. Color, upper surfaces and lower surfaces: Close to RHS163A.
   Presence of stipules.—Present. Frequency: Not at every bud.
   Stipule description.—Length: About 0.6 cm to about 1 cm. Width: About 0.3 cm. Shape: Triangular, very elongated. Margin: Erose.
Flower description:
   Flower type/habit.—3 to 6 flowers single flowers arranged on compound panicles. Flowers occur in general on one year old branches.
   Fragrance.—Weak, pleasant.
   Natural flowering season.—In April in south Germany, three days before ‘Weiroot 720’ (U.S. Plant Pat. No. 22,867).
   Lastingness of entire bloom period.—About 15 days.
   Lastingness of an individual bloom.—About 8 days.
   Inflorescence height.—About 4 cm to about 4.6 cm.
   Inflorescence diameter.—About 5 cm to about 9 cm.
   Flower diameter.—About 2.4 cm to about 3.1 cm.
   Flower depth.—About 1.2 cm to about 1.5 cm.
   Petals.—Arrangement: Five petals in a single whorl. Length: About 1.2 cm to about 1 cm. Width: About 1 cm to about 1.2 cm. Arrangement: Slightly overlapping. Shape: Oblong, slightly elongated. Apex: Rounded, slightly elongated. Base: Truncate. Margin: Entire. Texture, upper and lower surfaces: Smooth, glabrous. Color: When fully opened, upper surface: Close to RHS 155B. When opening and fully opened, lower surface: Close to RHS 155B.
   Sepals.—Arrangement: Five sepals in a single whorl. Length: About 0.4 cm. Width: About 0.3 cm. Shape: Triangular, moderately elongated. Apex: Pointed. Base: Truncate. Margin: Entire. Texture, upper and lower surfaces: Smooth, glabrous. Color, upper surfaces: Close to RHS 142B. Color, lower surfaces: Close to RHS 145B.
   Pedicels.—Length: About 2.3 cm to about 2.6 cm. Diameter: About 0.1 cm. Angle: About intermediate to semi-prostrate from the inflorescence stalk. Texture: Smooth, glabrous. Color: Close to RHS 142A.
   Peduncle.—Length: About 0.1 cm. Diameter: About 0.2 cm Surface: Smooth. Color: Close to RHS 149A.
   Reproductive organs.—Stamens: Quantity per flower: Numerous. Filament length: About 5 mm to 12 mm. Filament color: Close to RHS 155B. Anther length: About 0.3 mm to about 1 mm. Anther color: Close to RHS 6A. Pollen color: Close to RHS 6D. Pistils: Quantity per flower: One. Pistil length: About 10 mm to about 13 mm. Style length: About 8 mm to about 11 mm. Style color: Close to RHS 150C. Stigma shape: Round. Stigma color: Close to RHS 149A.
   Fruit description.—Maturity when described. Date picking: July in south Germany, varies with climatic conditions: About the same time as ‘Weiroot 720’ or about three days later. Size: Small, about 1.3 cm to about 1.6 cm diameter. Average weight: about 1.5 g, varies slightly with fertility of the soil, amount of thinning and climatic conditions. Form: Globose, Slightly flat at the apex. Stem cavity: Slightly obcordate to flat. Color: RHS 40A.
   Peduncle description.—Size: Average length about 3.6 cm. Average diameter about 0.1 cm. Color: RHS 149A.
   Stone description.—Average length: about 0.8 cm. Average width: about 0.75 cm. Average thickness: about 0.66 cm. Form: Nearly globose. Base: Flat. Apex: Rounded. Color: RHS 164C when dry.
   Fruit use.—Not be used as a dessert fruit.
Temperature tolerance: Plants of the new Prunus have been observed to tolerate temperatures from about −20° C. to about 40° C.

What is claimed is:

1. A new and distinct cultivar of Prunus cerasus L.×P.×schmittii Rehder named ‘STO3,’ substantially as illustrated and described herein.
